(19) United States
(12) Patent Application Publication
(10) Pub. No.: US 2023/0132535 A1
(43) Pub. Date: May 4, 2023

(54) PROLIFERATION SUPPRESSING AGENT FOR CANINE CANCER CELLS

(71) Applicant: Japan Animal Referral Medical Center, Kawasaki-shi, Kanagawa (JP)

(72) Inventors: Kenichiro Crawford ONO, Kawasaki-shi, Kanagawa (JP); Hirofumi YAMASAKI, Kawasaki-shi, Kanagawa (JP)

(73) Assignee: Japan Animal Referral Medical Center, Kawasaki-shi, Kanagawa (JP)

(21) Appl. No.: 17/916,025

(22) PCT Filed: Aug. 30, 2021
(86) PCT No.: PCT/JP2021/031802

§ 371 Date: Sep. 29, 2022

(30) Foreign Application Priority Data
Sep. 1, 2020 (JP) 2020-147007

(51) Int. Cl. A61K 31/517; A61K 45/06; A61P 35/00

(52) U.S. Cl. CPC A61K 31/517; A61K 45/06; A61P 35/00

(57) ABSTRACT

A proliferation inhibitor for canine cancer cells contains, as an active ingredient, 1-(3-((6,7-dimethoxyquinazolin-4-yl)oxy)phenyl)-3-(5-(1,1,1-trifluoro-2-methylpropan-2-yl)isooxazol-3-yl)urea or CEP-32496, and a method for suppressing proliferation of canine cancer cells. The cancer cells are caused by BRAF gene mutation. The canine cancer is canine transitional epithelial cancer, canine prostate cancer, canine malignant melanoma, or canine peripheral schwannoma.

### TECHNICAL FIELD

This present invention relates to a proliferation suppressing agent for canine cancer cells and the like. In particularly this present invention relates to a proliferation suppressing agent for canine cancer cells due to canine BRAF gene mutation (V595E: equivalent to human V600E) and the like.

### BACKGROUND ART

Transitional epithelial cancer having BRAF gene mutation (V595E), one of canine malignant tumors, is a tumor that is highly locally infiltrative and distantly metastatic, and early diagnosis and early therapy are desired. From the research results so far, it is known that V595E is expressed at a high rate (60-70%) in transitional epithelial cancer, and is a driver mutation that activates MAPK signaling pathway and makes transitional epithelial cells cancerous.

Activation of the MAPK signaling pathway by wild-type BRAF requires first activation of upstream Receptor tyrosine kinase (RKTs) and then phosphorylation of a BRAF kinase domain by RAS-GTP activation (Thr598/Ser601). This activated BRAF forms homodimers or heterodimers of ARAF, BRAF, and CRAF (including a B/CRAF-MEK complex) to phosphorylate and activate MEK. On the other hand, carcinogenic gene mutations have been observed in more than 30 sites in the BRAF kinase domain. Among these mutations, expression of V600 (such as V600E) is classified into mutation that forms RAS-independent monomers or mutation that forms dimers, and mutation that is expressed except for V600 and activate CRAF.

For cancer cells (such as malignant melanoma, colon cancer, and thyroid cancer) having human BRAF gene mutation (V600E), various BRAF inhibiting agents have been developed and used for therapy as cell proliferation suppressing agents and anti-tumor drugs. However, details such as canceration mechanisms such as dimerization of RAF, action mechanisms of the BRAF inhibiting agent (such as steric structures), or drug resistance that is expressed when the BRAF inhibiting agent is administered are clarified, and further development of inhibiting agents is desired and is being promoted.

That is, GDC0879 and AZ628, first generation BRAF inhibiting agents, suppress cell proliferation and canceration by inhibiting phosphorylation activity of BRAF (activation (phosphorylation) of MEK and ERK) as a small molecular substance that competes for ATP binding (small molecular ATP competitive BRAF inhibitor) at L-loop site (ATP-binding site) of BRAF activated by gene mutation.

However, as the diversity of BRAF gene mutations becomes clearer, the first-generation inhibiting agents have a limited anti-tumor effect on the BRAF mutations, and LPX4720, Vemurafenib, Dabrafenib and the like, second generation inhibiting agents targeting inhibition of BRAFV600E activity, cause drug resistance relatively early after administration.

On the other hand, none of therapeutic drugs (chemotherapeutic drugs) for canine transitional epithelial cancer show remarkable effect, and only the anti-tumor effect of oxicam-based non-steroidal anti-inflammatory drugs (NSAIDs) is expected. Canine tumors caused by canine BRAF gene mutation (V595E) have been clarified in transitional epithelial cancer, prostate cancer, malignant melanoma, peripheral schwannoma and the like. However, no BRAF inhibiting agents have been found as proliferation suppressing agents or therapeutic drugs for those cancer cells. In addition, since V595E is driver mutation, Vemurafenib, a typical second generation BRAF inhibiting agent, was investigated as a therapeutic drug for transitional epithelial cancer. However, in Vemurafenib, cell proliferation suppressing activity (IC50) is 8 micro M, and sufficient activity has not been obtained (Decker B et al. 2016). Since V595E is responsible gene mutation in canine cancer cells due to BRAFV595E, it is strongly desired to develop a useful BRAF inhibiting agent.

### PRIOR ART DOCUMENT

### Non-Patent Document

Non-Patent Literature 1: Decker B, Parker H G, Dhawan D et al. Mol. Cancer Res 2015; 13(6); 993-1002.

### SUMMARY OF THE INVENTION

### Problems to be Solved by the Invention

An object of this present invention is to provide a proliferation suppressing agent for canine cancer cells, in particular, a proliferation suppressing agent for canine cancer cells due to canine BRAF gene mutation (V595E) and the like.

### Means to Solve the Problems

This invention described herein is basically based on the findings of the example that the use of CEP-32496 may suppress proliferation of canine cancer cells due to BRAF gene mutation.

This invention described herein relates to a proliferation suppressing agent for canine cancer cells (the agent of the invention). The proliferation suppressing agent for canine cancer cells is an agent that is administered to target canines to suppress proliferation of canine cancer cells.

This agent includes 1-(3-((6,7-dimethoxyquinazolin-4-yl)oxy)phenyl)-3-(5-(1,1,1-trifluoro-2-methylpropan-2-yl)isoxazol-3-yl)urea (the compound of the invention) or CEP-32496 as an active ingredient.

CEP-32496 is synonymous with Agerafenib (INN), RXDX-105, AB-024, and AC-013773, and is commercially available as a research reagent for inhibiting agents against the human BRAF gene mutation (V600E). The active ingredient of CEP-32496 is the compound of the present invention described above.

An example of canine cancer cells is cancer cells caused by canine BRAF gene mutation (V595E). That is, an example of the above agents is a therapeutic agent for canine cancer. And examples of canine cancers are canine transitional epithelial cancer, canine prostate cancer, canine malignant melanoma, and canine peripheral schwannoma.

This description also provides a combination drug to be administered to canines (a combination drug for canine BRAF inhibiting agent-resistant cancer cells: the combination drug of the invention), along with other therapeutic drugs for cancer.

The agents of the present invention and the combination drugs of the present invention contain an effective amount of the compound of the present invention or CEP-32496 as an active ingredient. The agents of the present invention and the combination drugs of the present invention may be administered in an appropriate amount and at an appropriate frequency according to the body weight, age, and sex of the canines to be administered, administration method, and dosage form. As shown by the example, the above active ingredients are effective even in very small amounts. Therefore, an example of a single dose is 1 ng or more and 1 g or less, may be 10 ng or more and 500 mg or less, may be 100 ng or more and 100 mg or less, may be 1 mg or more and 100 mg or less, may be 1 mg or more and 20 mg or less per kg of the body weight. The frequency of administration may be, for example, once a week, once a day, twice or three times a day.

The agents of the present invention and the combination drugs of the present invention may be administered as an oral administration agent, a drink agent, a tablet agent, or an injectable agent. In injectable agents, the active ingredient may be administered intravenously, intramuscularly, subcutaneously or the like. Among these, intravenous administration is more preferable. For intravenous administration, the injectable agent may be administered directly to a subject from a syringe, or it is added first to a drip-feed solution in a drip bag and the subject may be given an intravenous drip.

If the agents of the present invention and the combination drugs of the present invention are injectable agents or drink agents, they may contain various materials such as water, saline, solvents, culture supernatants, pH adjusters, antioxidants (vitamin C), and trehalose, in addition to the active ingredients. If the agents of the present invention and the combination drugs of the present invention are tablet agents, they may contain various materials such as excipients and carriers.

This description also discloses a method of suppressing the proliferation for canine cancer cells and a therapeutic method for canine cancer, including the step of administering the agents of the present invention and the combination drugs of the present invention to target canines. An example of a canine cancer cell is the cancer cell caused by canine BRAF gene mutation (V595E). And examples of canine cancers are canine transitional epithelial cancer, canine prostate cancer, canine malignant melanoma, and canine peripheral schwannoma.

### Effects of the Invention

This present invention may provide a proliferation suppressing agent for canine cancer cells, in particular, a proliferation suppressing agent for canine cancer cells due to canine BRAF gene mutation (V595E) and the like.

### BRIEF DESCRIPTION OF THE DRAWINGS

FIG. 1 is a graph that replaces the drawing showing one example of the reaction curve of a BRAF inhibiting agent against V595E transitional epithelial cancer cells (TCCV-So strain). Only CEP32496 showed proliferation suppressing activity (IC50: 15 nM).

FIG. 2 is a table that summarizes proliferation suppressing activity of eight BRAF inhibiting agents against V595E transitional epithelial cancer cells (5 strains), taken from clinical cases and cloned in approximately 80 passages. Compared to the other 7 BRAF inhibiting agents, CEP32496 showed significantly higher activity.

FIG. 3 is a graph that replaces the drawing showing one example of the proliferation suppressing reaction curve at the time of simultaneous administration of CEP32496 and Vemurafenib in V595E transitional epithelial cancer cells (TCCV-Ka strain). The cell survival rate at the time of the simultaneous administration was clearly lower than that at the time of single agent administration, indicating a synergistic effect or additive effect.

FIG. 4 is a table that summarizes the IC50 values of V595E transitional epithelial cancer cells (5 strains), including one example (TCCV-Ka) shown in FIG. 3. In both established cell lines, the cell proliferation suppressing activity was enhanced when CEP32496 was simultaneously administered with Vemrafenib. In addition to competitive antagonism at the ATP-binding site of BRAF, which is the basis of BRAF inhibiting agent, CEP32496 was thought to exhibit BRAF inhibiting action by a mechanism other than the competitive antagonism.

FIG. 5 is a graph that shows one example of tumor formation during SCID mouse xenograft of V595E transitional epithelial cancer established cell lines (TCCV-Ka strain). In FIG. 5, 1×10⁷ cells/200 μl of established cell lines were inoculated subcutaneously on the ventral side of SCID mice (3 mice), and the formation of tumors was observed. As the target of anti-tumor activity, TCCV-Ka established cell lines were suitable from the viewpoint of oncogenicity, variation within individuals, growth tendency and the like. The three different symbols in the graph indicate each individual of the three mice.

FIG. 6 is a graph that showed the anti-tumor activity of CEP32496 by a xenograft test system. Significant suppression of tumor formation was observed from day 5 of CEP32496 administration, and CEP32496 showed the anti-tumor activity against V595E transitional epithelial cancer cells.

### DETAILED DESCRIPTION OF THE INVENTION

Hereinafter, embodiments of the present invention will be described. The present invention is not limited to the embodiments described below, but also includes those appropriately modified from the following embodiments to the extent apparent to those skilled in the art.

### Examples

Using five canine V595E transitional epithelial cancer established cell lines, taken from clinical cases by the inventors so far and cloned in approximately 80 passages, the cell proliferation suppressing activity of eight human BRAF inhibiting agents (GDC0879, AZ628, PLX4720, Encorafenib, Vemurafenib, Dabrafenib, RAF265, and CEP32496), which are available and have high cell proliferation suppressing activity, was investigated. In addition, in the xenograft test system, suppression of tumor growth (anti-tumor activity) was measured and the anti-tumor activity was investigated.

Cell Proliferation Suppressing Test

1,000 to 3,000 established cell lines (TCCV-Ka, TCCV-So, TCCV-Ec, TCCV-Ni, and TCCV-Ic) per well were spread on 96 well culture plates and cultured for 24 hours, then 8 BRAF inhibiting agents (GDC0879, AZ628, PLX4720, Encorafenib, Vemurafenib, Dabrafenib, RAF265, and CEP32496) dissolved in 0.1% DMSO were added with GDC0879, AZ628, Vemurafenib, Dabrafenib, and RAF265 at 0, 50, 100, 500, 1000, 5000, and 10000 nM, PLX4720 at 0, 1, 10, 100, 1000, 5000, and 10000 nM, Encorafenib at 0, 32, 63, 125, 250, 500, and 1000 nM, CEP32496 at 0, 3, 10, 30, 100, 300, and 1000 nM, the number of surviving cells after 24, 48, and 72 hours was measured using WST 1, and the cell proliferation suppressing activity was calculated.

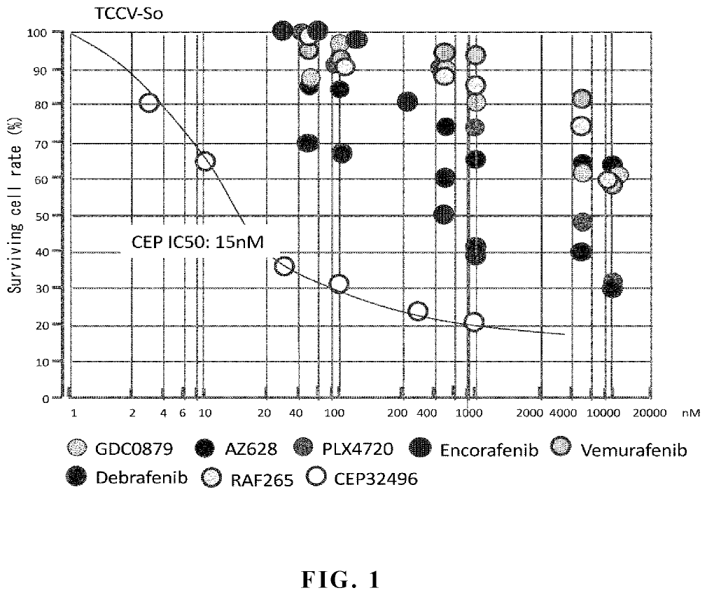

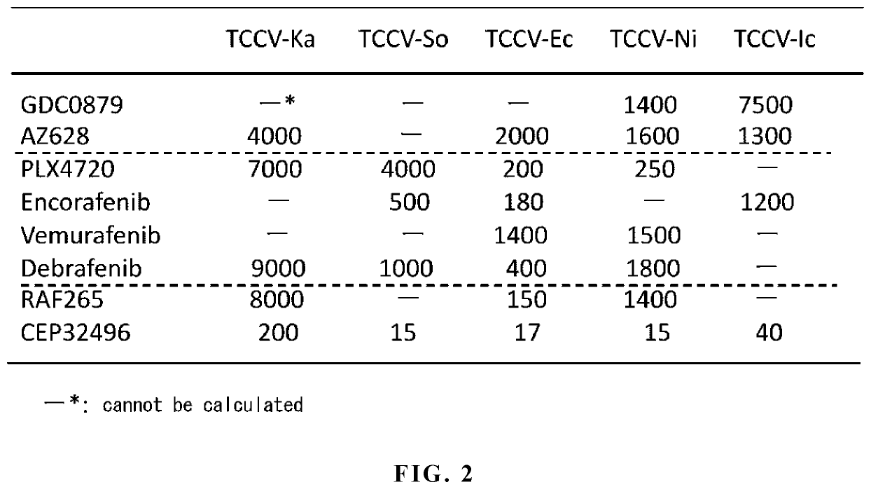

One example of the reaction curve of the BRAF inhibiting agent against canine V595E transitional epithelial cancer cells (TCC V-So strain) was shown in FIG. 1. In addition, the cell proliferation suppressing activity (IC50: nM) calculated from this reaction curve was shown in FIG. 2.

Four BRAF inhibiting agent, GDC0879, AZ628, Vemurafenib, and Dabrafenib, did not show sufficient proliferation suppressing activity (IC50: 200 nM or less) against all the cells used. For PLX4720, Encorafenib, and RAF265, IC50s of 200 nM, 180 nM, and 150 nM might be calculated only for the TCCV-Ec strain. On the other hand, the IC50 of CEP32496 might be calculated for all the cell strains used, and showed values of 9 to 200 nM. Therefore, only CEP32496 among the eight BRAF inhibiting agents was considered to be useful as a cell proliferation suppressing agent or a therapeutic drug for canine V595E transitional epithelial cancer.

The BRAF inhibiting agents, which are activated by V595E mutation, inhibit BRAF activity as a small molecule competitor for ATP binding (small molecular ATP competitive BRAF inhibitor) at the L-loop site of BRAF (ATP-binding site), and inhibit activation (phosphorylation) of MEK and ERK to suppress cell proliferation and canceration (Holderfield M, Nagel T E, Stuart D D. Br J Cancer 2014: 111(4): 640-645). Then, the CEP32496 cell proliferation suppressing mechanism was investigated by administering Vemurafenib, a typical BRAF inhibiting agent (second generation), in combination.

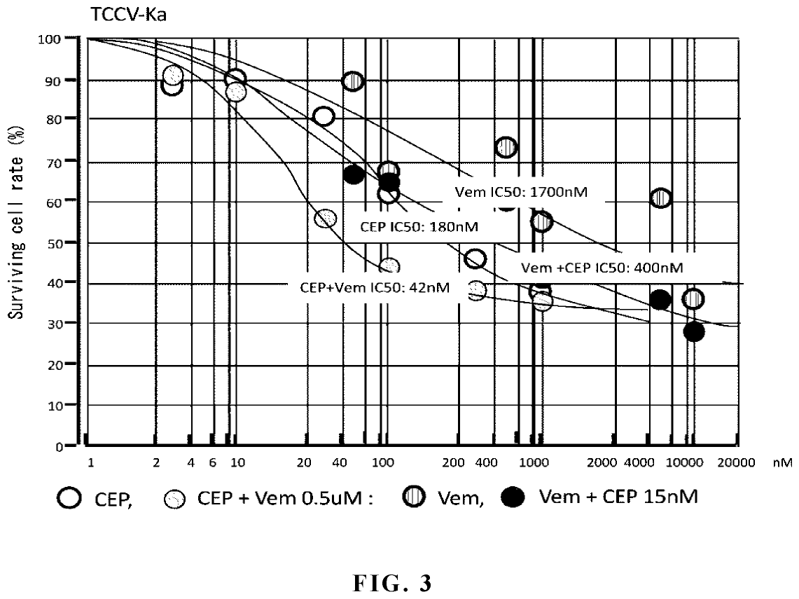

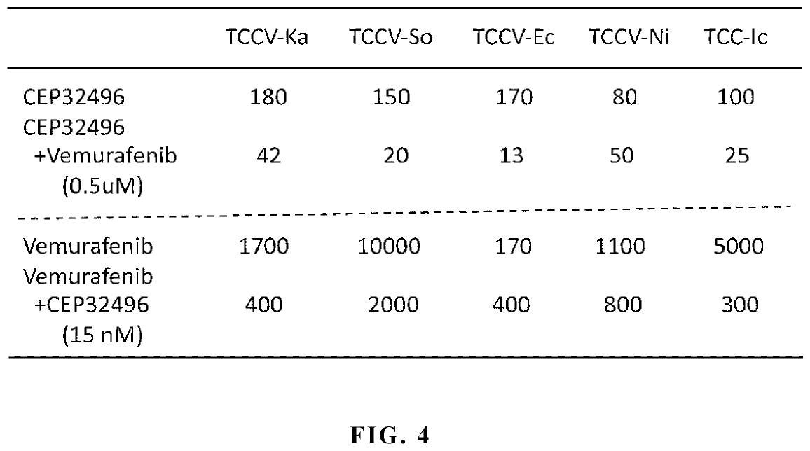

½ of the IC50 (1.5 μM) of Vemurafenib and CEP32496 obtained from TCCV-Ni strain, and conversely, ½ of the IC50 (15 nM) of CEP32496 and Vemurafenib obtained from TCCV-So strain were simultaneously administered to measure the cell proliferation suppressing activity against V595E transitional epithelial cancer cells (5 strains). In FIG. 3, one example of the reaction curve (TCCV-Ka strain) was shown. In addition, the calculated IC50 of V595E transitional epithelial cancer cells (5 strains) was shown in FIG. 4.

When Vemurafenib, which is a BRAF inhibiting agent and competitively inhibits binding to ATP, was co-administered with CEP32496, the cell survival rate was clearly lower than that when CEP32496 alone was administered (FIGS. 3 and 4). In addition, on the contrary, when CEP32496 was co-administered with Vemurafenib, the cell survival rate at the co-administration was clearly lower than that when Vemurafenib alone was administered. Therefore, in addition to competitive antagonism at the ATP-binding site of BRAF, which is the basis of BRAF inhibiting agent, CEP32496 was found to exhibit BRAF inhibiting action by a mechanism other than the competitive antagonism. In fact, CEP32496 has been reported to be a BRAF inhibiting agent, as well as inhibit MEK and ERK activities and exhibit multi-kinase properties (James J, Ruggen B, Armstrong R C et al. Mol Cancer Ther 2012; 11(4): 930-941).

Anti-Tumor Activity of CEP32496 by a Xenograft Test System

V595E Transitional Epithelial Cancer Cells Used for a Graft

In order to observe the anti-tumor activity in the xenograft test system, it has been reported that the tumor growth depends on the number of cells inoculated, cell strains, and animal species. Then, five strains of V595E transitional epithelial cancer established cell lines, 1×10⁶ and 1×10⁷ cells/200 μl, were inoculated subcutaneously on the ventral side of SCID mouse (CB17/Icr-scid/scid. Jcl, CLEA Japan), and the formation of tumors was observed.

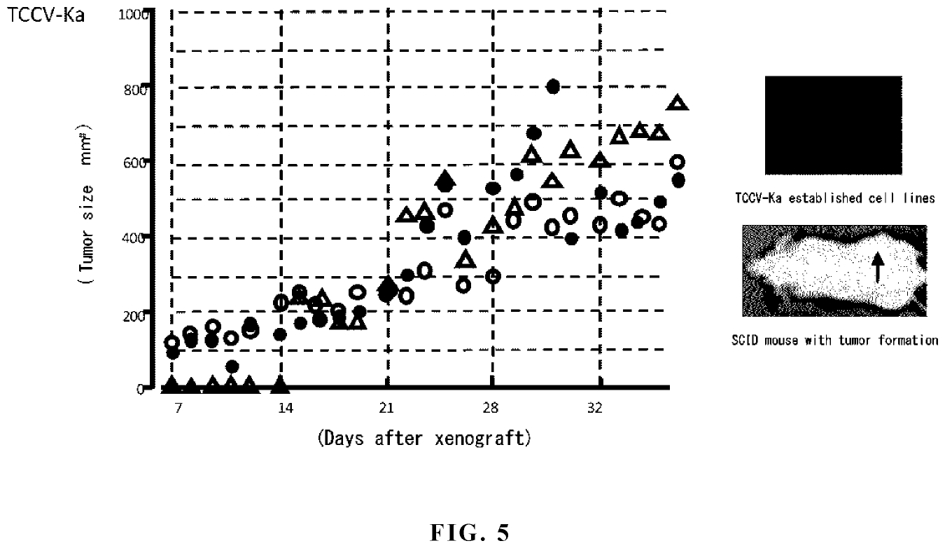

After about one month of observation, tumor formation (4×4 mm or more: oncogenicity) was observed in all established cell lines when inoculated with a solution of 1×10⁷ cells suspended in 300 μl of PBS. However, after that, there were many strains that did not grow (TCCV-Ni strain), disappeared (TCCV-Ic strain), and grow but were not suitable as a control for observing the anti-tumor activity due to their large fluctuation range. FIG. 5 showed the tumor formation curve of the TCCV-Ka strain, which is suitable as a control for determining the anti-tumor activity of CEP32496.

Anti-Tumor Activity of CEP32496

Using the TCCV-Ka strain as a graft cell, 1×10⁷ cells were inoculated on the right ventral side of 5 SCID mice in each of the administration group and the control group. Two weeks later, a tumor of approximately 5×5 mm was formed. In the control group, 10% DMSO was administered intraperitoneally, and in the administration group, 200 μl of CEP32496 suspension solution (10 mg/kg) in 10% DMSO was administered intraperitoneally. The administration was 5 times/week.

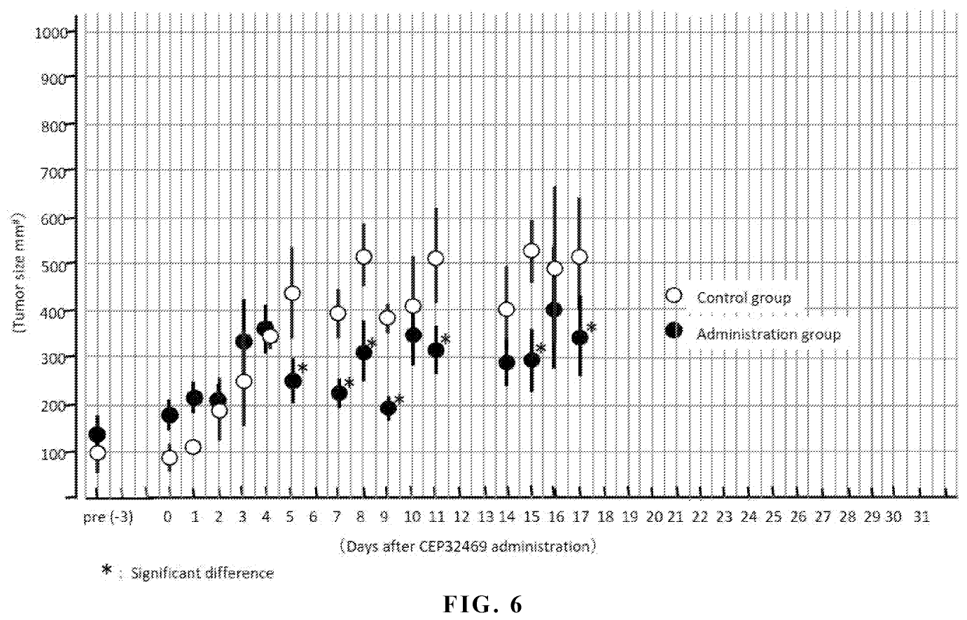

The CEP32496 (10 mg/kg) administration group showed significant suppression of tumor formation after 5 days of administration compared to the control group, and showed anti-tumor activity against BRAFV595E transitional epithelial cancer cells (FIG. 6).

### DISCUSSION

The usefulness of CEP32496 was clarified as a cell proliferation suppressing agent and as an anti-tumor drug against cancer cells due to canine BRAF gene mutation (V595E), for which no accurate therapeutic method has been confirmed so far. It is considered that a new therapeutic method for these tumors may be developed by early diagnosis by genetic diagnosis and the like, and administration of CEP32496.

### INDUSTRIAL APPLICABILITY

This invention may be utilized in the field of drugs for animals.

1. A proliferation suppressing agent for canine cancer cells, comprising: 1-(3-((6,7-dimethoxyquinazolin-4-yl)oxy)phenyl)-3-(5-(1,1,1-trifluoro-2-methylpropan-2-yl)isoxazol-3-yl)urea or CEP-32496 as an active ingredient.

2. The agent according to claim 1, wherein the canine cancer cells are due to BRAF gene mutation.

3. The agent according to claim 1, wherein the agent is a therapeutic agent for canine cancer.

4. The agent according to claim 3, wherein the canine cancer is canine transitional epithelial cancer, canine prostate cancer, canine malignant melanoma, or canine peripheral schwannoma.

5. The agent according to claim 1, wherein the agent is a combination drug for canine BRAF inhibiting agent-resistant cancer cells.

6. A method for suppressing proliferation of canine cancer cells, comprising:
administering to a subject in need thereof a proliferation suppressing agent comprising 1-(3-((6,7-dimethoxyquinazolin-4-yl)oxy)phenyl)-3-(5-(1,1,1-trifluoro-2-methylpropan-2-yl)isoxazol-3-yl)urea or CEP-32496.

7. The method according to claim 6, wherein the canine cancer cells are due to BRAF gene mutation.

8. The method according to claim 6, wherein the proliferation suppressing agent is a therapeutic agent for canine cancer.

9. The method according to claim 8, wherein the canine cancer is canine transitional epithelial cancer, canine prostate cancer, canine malignant melanoma, or canine peripheral schwannoma.

10. The method according to claim 6, wherein the proliferation suppressing agent is a combination drug for canine BRAF inhibiting agent-resistant cancer cells.
